(19) United States
(12) Patent Application Publication
(10) Pub. No.: US 2007/0134319 A1
(43) Pub. Date: Jun. 14, 2007

(54) Pharmaceutical formulations of bisphosphonates

(75) Inventors: Erika Aina Zannou, Edison, NJ (US); Simon David Bateman, Randolph, NJ (US); Madhusudhan Pudipeddi, Edison, NJ (US); Alan Edward Royce, Saylorsburg, PA (US); Abu T.M. Serajuddin, Queens, NY (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 10/583,646

(22) PCT Filed: Dec. 22, 2004
(86) PCT No.: PCT/EP04/14645

§ 371 Date: Aug. 15, 2006

Related U.S. Application Data
(60) Provisional application No. 60/532,334, filed on Dec. 23, 2003.

(51) Int. Cl. A61K 9/20; A61K 31/66

(52) U.S. Cl. 424/464; 514/102

(57) ABSTRACT

A pharmaceutical formulation comprising an oral dosage form containing a bisphosphonic acid or a salt thereof and an inactive ingredient selected from: an ester of a medium chain fatty acid, or a lipophilic polyethylene glycol ester, said inactive ingredient having a hydrophilic lipophilic balance (HLB) of from about 1 to about 30.

This invention relates to the use and preparation of pharmaceutical forms of bisphosphonates, in particular to oral pharmaceutical formulations of bisphosphonates. The invention is useful in the preparation of oral pharmaceutical forms of bisphosphonates and the treatment of conditions of abnormally increased bone turnover, including osteoporosis and hypercalcemia resulting from excessive bone resorption secondary to hyperparathyroidism, thyrotoxicosis, sarcoidosis, or hypervitaminosis D.

Bisphosphonates show activity which is useful, in vertebrate animals, for those conditions which exhibit or are initiated by abnormal bone turnover. Bisphosphonates are widely used to inhibit osteoclast activity in a variety of both benign and malignant diseases in which bone resorption is increased. Thus, bisphosphonates have recently become available for long-term treatment of patients with Multiple Myeloma (MM). These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of Paget's disease of bone, tumour-induced hypercalcemia and, more recently, bone metastasis and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still poorly understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit both the activation and the activity of osteoclasts.

Oral dosing of bisphosphonates typically presents significant hurdles since oral administration of bisphosphonates can be corrosive to the gastrointestinal tract. Bisphosphonates thus tend to produce adverse gastric disturbances in animals and man. The adverse gastric disturbances caused by orally dosed bisphosphonates may result in nausea, vomiting, diarrhea, bloody discharge, and ulcerations, even to the point where emergency medical interventions are required. Those bisphosphonates which are marketed to be dosed orally typically have dosing regimens which must be closely followed by patients in order to afford minimal gastric disturbances and erosive effects. In addition the bisphosphonates which are marketed typically demonstarte low gastric absorption and resulting bioavailability. Thus, an effective oral dose amount of the marketed bisphosphonates in present formulations typically requires quantities of the bisphosphonate which may cause gastric disturbances. Specific dosing regimens may be employed to enable adequate absorption and increase tolerability of an orally dosed bisphosphonate, for example, see product labelling for FOSAMAX (alendronate sodium) in the Physician's Desk Reference, 2003 edition, Thomson Healthcare, Montvale, N.J. 07645. However, the present oral dosing regimens pose significant compliance obstacles, particularly in the elderly population for which such bisphosphonates are prescribed and also allow for the chance that non-adherence to the exact regimen may lead to gastric ulceration or more severe effects. Even adherence to the relatively complicated dosing regimes may lead to gastric disturbances and ulcerations in susceptible individuals in part because of the amount of a bisphosphonate required to be orally dosed in order to overcome its low oral route bioavailability. In the present invention, the additional use of various inactive agents as elements which increase gastric absorption and/or protect the gastrointestinal tract from chemical and/or mechanical damage induced by the bisphosphonates (hereinafter referred to as the active agents of the invention), may allow the oral effective dose of a bisphosphonate to be reduced to a level which significantly reduces its gastric side effects and enables treatment of a much broader population of patients than with present formulations. Thus, the present invention provides a means to overcome oral dosing obstacles with a more patient friendly formulation of an active agent, particularly bisphosphonates, that is gastrically compatible and/or optimally bioavailable with respect to oral compositions which are presently available.

A balance between tolerability and bioavailability is sought for the composition of the present invention. A formulation which is very bioavailable may not necessarily be gastrically compatible. Optimal bioavailability allows therapeutically relevant blood levels of active agent to be achieved with oral dosing and is associated with a decreased level of gastric clinical toxicological signs in the dosed subject as compared to present or conventional oral formulations of the active agents of the invention, such as bisphosphonates.

Conditions of abnormally increased bone turnover which may be treated in accordance with the present invention include: treatment of postmenopausal osteoporosis, e.g. to reduce the risk of osteoporotic fractures; prevention of postmenopausal osteoporosis, e.g. prevention of postmenopausal bone loss; treatment or prevention of male osteoporosis; treatment or prevention of corticosteroid-induced osteoporosis and other forms of bone loss secondary to or due to medication, e.g. diphenylhydantoin, thyroid hormone replacement therapy; treatment or prevention of bone loss associated with immobilisation and space flight; treatment or prevention of bone loss associated with rheumatoid arthritis, osteogenesis imperfecta, hyperthyroidism, anorexia nervosa, organ transplantation, joint prosthesis loosening, and other medical conditions. For example, such other medical conditions may include treatment or prevention of periarticular bone erosions in rheumatoid arthritis; treatment of osteoarthritis, e.g. prevention/treatment of subchondral osteosclerosis, subchondral bone cysts, osteophyte formation; treatment or prevention of hypercalcemia resulting from excessive bone resorption secondary to hyperparathyroidism, thyrotoxicosis, sarcoidosis, and hypervitaminosis D.

It is contemplated that the pharmaceutical compositions of the present invention may be, for example, compositions for enteral, such as oral, rectal, aerosol inhalation or nasal administration,and parenteral, such as intravenous or subcutaneous administration.

Interesting results are achieved with compositions of the present invention which are adapted to oral administration. Orally administrable pharmaceutical preparations are dry-filled hard or soft capsules for example, made of gelatin, hydroxypropylmethylcellulose (HPMC), a starch derivative and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as aqueous buffer solutions to dissolve the bisphosphonate or fatty oils, paraffin oil or liquid polyethylene glycols, to aid suspension or dissolution in the inactive ingredients, it being possible also for stabilisers to be added. Interesting results are achieved when semi-solid fatty acid glycerides, such as for example, GELUCIRE® (lauroyl macrogol-32 glycerides, Gatefosse, Westwood, N.J.) or semi-solid lipid based bioavailability enhancers such as VITAMIN E-TPGS (water soluble D-alpha-tocopheryl polyethylene glycol 1000 succinate, Peboc Division of Eastman Chemicals, Anglesey, UK) may be used as a melt, semi-solid or liquid solution or suspension filled into hard or soft capsules made of gelatin, HPMC or starch derivatives.

It is counterintuitive that such inactive ingredients would increase the bioavailability of a readily water-soluble active ingredient such as a bisphosphonate. It is also novel that such inactive ingredients would increase the oral tolerability and/or inhibit the gastric damage resulting from orally dosed bisphosphonates. Thus, the utility of such fatty acid glyceride and amphipathic inactive ingredients, in the present invention, is curious and novel. In addition, the use and benefit of such inactive ingredients, for example, GELUCIRE® and VITAMIN E-TPGS in oral formulations of bisphosphonates is not identfied in the prior art. Gelucire® 44/14 is synthesized by an alcoholysis/esterification reaction, using hydrogenated palm kernel oil and PEG 1500 as starting materials. GELUCIRE 44/14 is therefore a well-defined mixture of mono-, di-and triglycerides and mono- and di-fatty acid esters of polyethylene glycol. The predominant fatty acid is lauric acid (C12). Gelucire® 50/13 is synthesized by an alcoholysis/esterification reaction using hydrogenated palm oil and PEG 1500 as starting materials.

Gelucire® 50/13 is therefore a well defined mixture of mono-,di-and triglycerides and mono- and di-fatty acid esters of polyethylene glycol. The predominant fatty acid is palmitostearic acid (C16-C18).

Pharmaceutical preparations for enteral and parenteral administration are, for example, those in dosage unit forms, such as dragees, tablets, soft or hard gelatin capsules and also ampoules. They are prepared in a manner known per se, for example by means of conventional mixing, granulating, confectioning, dissolving, melting or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or drag\acute{ecores.}

Suitable carriers may be fillers, such as sugars, for example lactose, saccharose, mannitol or sorbitol, cellulose preparations and/or calcium phosphates, for example tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Colouring substances or pigments may be added to the tablets or dragee coatings, for example for the purpose of identification or to indicate different doses of active ingredient.

The particular mode of administration and the dosage may be selected by the attending physician taking into account the particulars of the patient, especially age, weight, life style, activity level, hormonal status (e.g. post-menopausal) and bone mineral density as appropriate.

The dosage of the active agents of the Invention may depend on various factors, such as effectiveness and duration of action of the active ingredient, e.g. including the relative potency of the bisphosphonate used, mode of administration, warm-blooded species, and/or sex, age, weight and individual condition of the warm-blooded animal.

Normally the dosage is such that a single dose of the bisphosphonate active ingredient from 0.005-1000 mg/kg, and often 0.01-10 mg/kg, is administered to a warm-blooded animal weighing approximately 75 kg.

“mg/kg” means mg drug per kg body weight of the mammal—including man—to be treated.

The dose mentioned above is typically administered intermittently with a regular dosing interval of, for example, once a day, once a week, once a month, once every six months, once a year or less frequently as allowed in accord with the duration of therapeutic activity of an individual bisphosphonate.

Formulations in single dose unit form contain preferably from about 1% to about 90%, and formulations not in single dose unit form contain preferably from about 0.1% to about 20%, of the active ingredient. Single dose unit forms such as ampoules of infusion solution or solid for preparation of infusion solution doses, capsules, tablets or dragees contain e.g. from about 0.5 mg to about 2000 mg of the active ingredient. It will be appreciated that the actual unit dose used will depend upon the potency of the bisphosphonate and the dosing interval amongst other things. Thus the size of the unit dose is typically lower for more potent bisphosphonates and greater the longer the dosing interval. For example, for more potent, recent bisphosphonates such as zoledronic acid a unit dose of from about 0.5 up to about 2000 mg may be used. For example, also for such recent, more potent bisphosphonates a unit dose of from about 2 to about 200 mg may be used for dosing

Thus in the present description the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition. In certain embodiments the invention may be used for the prophylactic treatment of osteoporosis and similar diseases. Thus for example, bisphosphonates may be administered to individuals at risk of developing osteoporosis, such as for example, post-menopausal women, on a routine basis, at regular dosing intervals of, for example, once a day, once a week, once a month, once every six months, once a year or less frequently as allowed in accord with the duration of activity of an individual bisphosphonate. For example, it is disclosed in U.S. Patent application No. 60/267689, which patent application is herein incorporated by reference, that the bisphosphonate, zoledronic acid, for the treatment of osteoporosis, may be dosed at intervals of once very six months, once a year, up to once every three years or even less frequently.

The bisphosphonates used in the present invention are typically those which inhibit bone resorption.

Thus, for example, suitable bisphosphonates for use in the composition of the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 1-(4-chlorophenylthio)methane-1,1-diphosphonic acid (tiludronic acid), e.g. tiludronate; 3-[N-(2-phenylthioethyl)-N-methylamino]-1-hydroxypropane-1,1-di-phosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), and YM175.

Pharmaceutically acceptable salts of the active agents which have at least some clinically useful amount of chemical stability, therapeutic efficacy, and gastric absorption and tolerance may be salts with bases, conveniently metal salts derived from groups Ia, lb, Ia and IIb of the Periodic Table of the Elements, including alkali metal salts, e.g. potassium and sodium salts, or alkaline earth metal salts. For example, interesting results have been achieved with calcium or magnesium salts, and also ammonium salts with ammonia or organic amines and salts wherein one, two, three or four, in particular one or two, of the acidic hydrogens of the bisphosphonic acid are replaced by a pharmaceutically acceptable cation, as seen in the case of sodium, potassium or ammonium salts, notably in sodium, and also in salts characterized by having one acidic hydrogen and one pharmaceutically acceptable cation, for example sodium, in each of the phosphonic acid groups.

All the bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130.

The Active Agents of the Invention may be used in the fonn of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The Active Agents of the Invention can also be used in the form of their hydrates or include other solvents used for their crystallisation.

The Active Agents of the Invention (the bisphosphonates) are preferably used in the form of pharmaceutical compositions that contain a therapeutically effective amount of active ingredient optionally together with or in admixture with inorganic or organic, solid, semi-solid or liquid, pharmaceutically acceptable carriers which are suitable for administration.

The Active Agents of the Invention may be administered alone or in combination with other bone active drugs, either in fixed combinations or separately both physically and in time, such as hormones, e.g. estrogen, calcitonins, parathyroid hormone or analogues of any of these, raloxifene or other selective estrogen receptor modulators (SERMs). Such additional bone active drugs may be administered more frequently than the bisphosphonate.

### EXAMPLES

The following Examples illustrate the invention described hereinbefore and are not meant to limit the invention in any way.

In the following Example the term “active ingredient” is to be understood as being any one of the bisphosphonic acid derivatives and therapeutically effective salts and hydrates thereof mentioned above as being useful according to the present invention.

Tablet Formulation for Dog Studies

Formulations in Table 1 are tableted using a Carver press (Carver, Inc., Wabash, Ind.), with a compression pressure about 1 ton, into 10 mm bevelled edge tablets. Stearic acid is used as a lubricant to avoid potential complexation of zoledronic acid with Mg²⁺ upon dissolution. Citric acid is used to bring the pH of a dog's stomach closer to that of the human stomach pH and the composition of the invention may be formulated with or without citric acid to accommodate the tested species. The formulation containing citric acid may stick to the punches and require lubrification of the punches prior to compression. Addition of a surfactant to the formulation should be avoided since it may be irritating to the Gastro-intestinal tract (GIT).

TABLE 1
Example of a zoledronic acid conventional tablet with and without
citric acid.)}

| | With Citric Acid | | Without Citric Acid | |
|---|---|---|---|---|
| Ingredients | % | Amount (mg) | % | Amount (mg) |
| ZOL446 monohyd. | 30.5 | 106.6 | 30.5 | 106.6 |
| Citric acid anhyd. | 28.6 | 100.0 | | |
| Lactose DT | 16.0 | 56.0 | 60.5 | 211.9 |
| Emcocel 90M | 16.0 | 56.0 | 16.0 | 56.0 |
| Crospovidone | 5.0 | 17.5 | 5.0 | 17.5 |
| Stearic acid | 4.0 | 14.0 | 4.0 | 14.0 |
| Total | 100.0 | 350.0 | 100.0 | 350.0 |

Complete release of zoledronic acid in both types of conventional tablets is achieved in vitro in about 15 min. at pH 4.5 at 37° C., using dissolution apparatus paddles set at 50 rpm.

Lipid Sustained Release Formulation

The solubility of zoledronic acid in GELUCIRE 44/14 and GELUCIRE 50/13 is poor and is determined to be less than 1 mg/g at 60° C.

In a suspension, of the active agents of the Invention in GELUCIRE, the release of zoledronic acid will be sustained, therefore, the drug substance will be likely to readily solubilize when it comes in contact with the stomach contents. For homogeneity of the suspension and dissolution optimization, a reduced particule size zoledronic acid may be used. This may be formulated with or without citric acid. Citric acid is poorly soluble in GELUCIRE also and its particle size may be decreased using a mortar and pestle.

Capsules of size #0 may contain up to 660 and 680 mg of GELUCIRE 50/13and GELUCIRE 44/14, respectively. The formulation may conveniently be set at 500 mg GELUCIRE per 100 mg of zoledronic acid. Mixture of GELUCIRE are used for optimum release rate: GELUCIRE 55/13 alone may provide a 100% release in about 7-8 hours; 50:50 of GELUCIRE 50/13: GELUCIRE 44/14 may provide a 100% release of 3 to 3.5 hours.

Microemulsion Formulation I

Various liquid lipidic media, known as potential bioavailability enhancers may be considered, for example as shown in the list below. Zoledronic acid solubility in all the excipients of the Example is assessed visually and is determined to be minimal (<0.2 mg/g of excipient). A zoledronic acid formulation in one of these lipidic media is administered to the studied dogs by gavage. The formulation conveniently may have 5 mL of the potential bioavailability enhancer per 100 mg of zoledronic acid (20 mg/mL). Based on the poor solubility of zoledronic acid poor in the excipients, a formulation in the excipients will likely be a suspension. However it is possible that a formulation of the invention will also allow complete dissolution of the active agents in the excipients. A reduced particle size of about 200 micrometers zoledronic acid may be used to maximize the suspension homogeneity. The suspensions will typically sediment rapidly after preparation and will likely need to be prepared extemporaneously, prior to the administration to dogs.

The tested excipients and their composition are detailed below:
placebo
   43.0% CREMOPHOR (BASF, Ludwigshafen, Germany), 35.7% comoil-mono-di-tri-glyceride, 10.6% propylene glycol, 10.6% ethanol, 0.1% tocopherol DL-alpha
LABRASOL, (Gatefosse, Westwood, N.J.)
   Caprylocaproyl Macrogol-8 glycerides, HLB=14, used as bioavailability enhancer
LABRAFIL M2125CS, (Gatefosse, Westwood, N.J.)
   Linoleoyl Macrogol-6 glycerides, HLB=4, used as bioavailability enhancer
CAPROYL PGMC, (Gatefosse, Westwood, N.J.)
   Propylene glycol monocaprylate, HLB=5, used as solubilizer and absorption enhancer
CAPMUL® PG-8, (Abitec Corp., Janesville, Wis.)
   Propylene glycol monoester of medium chain fatty acids (primarily caprylic acid), HLB=4.4, non-toxic after 1000 and 2500 mg/kg administered in the Beagle dogs for 28 consecutive days, emulsifier/surfactant used as solubilizing agent and bioavailability enhancer, readily absorbed
CAPMUL MCM, (Abitec Corp., Janesville, Wis.)
   Medium chain mono- and diglyceride (primarily caprylic and capric acid), HLB=5.5-6.0, emulsifier/surfactant used as solubilizing agent and bioavailability enhancer, readily absorbed
CAPTEX 200, (Abitec Corp., Janesville, Wis.)
   Propylene glycol dicaprylate/dicaprate, used as bioavailability enhancer
CAPTEX 355 EP, (Abitec Corp., Janesville, Wis.)
   Caprylic/capric triglyceride, used as bioavailability enhancer
      VITAMIN E-TPGS Formulation

VITAMIN E-TPGS is a semi-solid excipient with a melting point of about 41° C. and hydrophilic lipophilic balance (HLB) of 15-19. It is readily absorbed from the gastro-intestinal tract (GIT).

The solubility of zoledronic acid is less than 0.22 mg/g of VITAMIN E-TPGS at 40° C. The VITAMIN E-TPGS capsules are prepared using zoledronic acid milled to a particle size of about 200 micrometers. This drug substance is suspended in VITAMIN E-TPGS which may be pre-heated to about 40° C. to form a dispersion. The dispersion may then be encapsulated. Dissolution of the drug substance is pH-independent. Apparent complete release from the gelatin capsules is achieved in about 75 min.

Formulation Selection for a Canine Study

Selection of Formulation and Mode of Administration

Formulations and mode of administration are selected from the formulations described above.
The dogs are randomized into 5 groups, one for each of the five formulations. Unit doses are prepared based on a dog's projected weight at the start of the study. Liquid formulations are administered by gavage (20 mg/mL zoledronic acid solution or suspension), semi-solid formulations are administered in gelatin capsules (0.2 mg/mg zoledronic acid suspension),
Four of the formulations are flushed with a citric acid solution, one of the formulations serves as a control for the citric acid effect and is flushed with tap water (flush of 2.5 mL/kg). The citric acid solutions concentration is based on the zoledronic acid dose: 24 mg/mL (pH˜2.2) for the 10 mg/kg dose and 60 mg/mL (pH˜2.1) for the 25 mg/kg dose. Rationale for the citric acid flush is: (a) lowering of the dogs' stomach pH, (b) solubilization of part of the calcium:zoledronic acid complexes that might form in situ, (c) potential enhancement of paracellular transport.
   Manufacture of the Formulations of the Examples

The five formulations for oral administration in the Example are as follows:
1. 20 mg/mL zoledronic acid solution in pH 4.5 acetate buffer with citric acid flush,
2. 20 mg/mL zoledronic acid solution in pH 4.5 acetate buffer with tap water flush,
3. 0.2 mg/mg zoledronic acid suspension in GELUCIRE with citric acid flush,
4. 20 mg/mL zoledronic acid suspension in CAPMUL PG-8 with citric acid flush,
5. 0.2 mg/mg zoledronic acid suspension in VITAMIN E-TPGS with citric acid flush.
   Formulations 1, 2 and 4 and Citric Acid Flush

Formulations 1, 2 and 4 are prepared in situ prior to administration to dogs. Prior to each administration, one formulation is prepared per group of dogs by addition of the excipient or buffer into the pre-weighed drug substance and agitation on a stir plate. The formulations are kept under constant agitation during administration. The dose is administered based on volume, corrected for each dog's weight.

Zoledronic acid is readily soluble in pH 4.5 acetate buffer and does not precipitate out upon addition of tap water (final pH 3.8) or citric acid (final pH˜2.2) flush. Also, zoledronic acid is readily dispersed homogeneously into CAPMUL PG-8. An emulsion forms upon addition of the citric acid flush to the CAPMUL PG-8 suspension, with complete solubilization of zoledronic acid after about 10 min. agitation.

Formulation 3

A formulation of zoledronic acid in GELUCIRE capsules is detailed in Table 2.

TABLE 2
zoledronic acid GELUCIRE formulation

| Formulation | 10 mg/kg | 25 mg/kg |
|---|---|---|
| (for a 10 kg dog) | (mg) | (mg) |
| Gelucire 50/13 | 250.0 | 625.0 |
| Gelucire 44/14 | 250.0 | 625.0 |
| zoledronic acid monohydrate | 106.6 | 266.5 |
| Capsule size | 0 | 000 |
| Fill Weight (mg) | 606.6 | 1516.5 |

GELUCIRE 44/14 is melted at 65-70° C. and weighed accurately. GELUCIRE 50/13 is then weighed and added to the melted GELUCIRE 44/14. The mixture is melted and homogenized at 65-70° C. Zoledronic acid having a particle size of about 200 micrometers is added slowly while stirring using a low shear mixer. The mixture is kept at 65-70° C. under constant stirring during capsule filling. Capsules are filled manually using a positive displacement pipet. Each capsule's content is accurately weighed based on the unit dose and each dog's weight. Capsules are placed at 40° C. for 36 hours for curing and are then refrigerated at 4-8° C. until administration.

The capsules are analyzed for zoledronic acid content and degradation products using high power liquid chromatography (HPLC). Example results are detailed below:
10 mg/kg strength: assay=97.9%,
25 mg/kg strength: assay=97.6%.
   Formulation 5

The formulation of the VITAMIN E-TPGS™ capsules is detailed in Table 3.

TABLE 3
Zoledronic acid VITAMIN E-TPGS formulation

| Formulation | 10 mg/kg | 25 mg/kg |
|---|---|---|
| (for a 10 kg dog) | (mg) | (mg) |
| Vitamin E-TPGS | 500.0 | 1250.0 |
| zol. monohydrate | 106.6 | 266.5 |
| Capsule size | 0 | 000 |
| Fill Weight (mg) | 606.6 | 1516.5 |

VITAMIN E-TPGS is melted at 50° C. Zoledronic acid of a particle size of about 200 micrometers is added slowly while stirring. The mixture is kept at 50° C. under constant stirring during capsule filling. Capsules are filled manually using a positive displacement pipet. Each capsule's content is accurately weighed based on the unit dose and each dog's weight. Capsules are kept at 4-8° C. until administration.

The capsules are analyzed for zoledronic acid content and degradation products by HPLC. Example results are detailed below:
10 mg/kg strength: assay=99.2%,
25 mg/kg strength: assay=99.6%.
   Dosing and Tolerability and Bioavailability Testing

Zoledronic acid formulations and dog study groups are prepared as described herein and above. The formulations are administered orally via gavage or capsule, once daily at doses of 10 or 25 mg zoledronic acid/kg/day, to five groups (3/dose/group) of fasted male beagle dogs for up to 1 week.

Formulation Subject Groups:

Groups 1 and 2: receive zoledronic acid as solutions of zoledronic acid in acetate buffer flushed with citric acid and tap water, respectively.
Group 3: receives zoledronic acid as a suspension in GELUCIRE which is placed in a gelatin capsule and is flushed with citric acid.
Group 4: receives a semi solid suspension of zoledronic acid in CAPMUL PG-8, flushed with citric acid.
Group 5: receives zoledronic acid as a suspension in VITAMIN E-TPGS which is placed in a gelatin capsule and is flushed with citric acid.

The dosing volumes for groups 1, 2 and 4 are 0.5 mL/kg (10 mg/kg/day) and 1.25 mL/kg (25 mg/kg/day).

Male beagle dogs may be procured from Marshall Farms, North Rose, New York. At the start of dosing, animals are approximately 7 to 9 months of age and body weights range from about 7. to about 10. kilograms. Clinical signs are collected daily (prior to dosing, within 5 minutes postdose, and at approximately 0.5, 1, 2, 4 and 6 hours postdose). Body weight and food consumption determinations are conducted on all groups. Bioavailability may be determined by HPLC analysis for zoledronic acid levels in serum samples collected from all animals at approximately 24 hours following the first and seventh doses. Blood samples may be collected for toxicokinetic analyses from moribund animals prior to sacrifice and from surviving animals after the first and seventh daily dose and prior to sacrifice. Necropsies may be performed on all animals and macroscopic findings are recorded.

(a)

TABLE 4
Study design, animal allocation and test article
doses

| | | Dose* | Concentration** | Dose |
|---|---|---|---|---|
| Group | Number | (mg/kg/day) | (mg/mL) | volume |
| Formulation 1 | 3 | 10 | 20 | 0.5 |
| | 3 | 25 | 20 | 1.25 |
| Formulation 2 | 3 | 10 | 20 | 0.5 |
| | 3 | 25 | 20 | 1.25 |
| Formulation 3 | 3 | 10 | NA | NA |
| | 3 | 25 | NA | NA |
| Formulation 4 | 3 | 10 | 20 | 0.5 |
| | 3 | 25 | 20 | 1.25 |
| Formulation 5 | 3 | 10 | NA | NA |
| | 3 | 25 | NA | NA |

NA = not applicable

Results

At 10 mg/kg/day, test article-related moribundity occurred in 1 dog receiving formulation 1 and in all dogs receiving formulation 4. At 25 mg/kg/day, test article-related moribundity occured in all dogs receiving formulations 1, 3 and 4; in two dogs receiving formulation 2 an in 1 dog receiving formulation5. Moribundity was observed as early as day 4 in animals receiving formulation 4 at doses of 10 and 25 mg/kg/day while dogs in the other dose groups were sacrificed moribund on day 6 or 7. The cause of death or moribundity in the animals that died or were sacrificed early was due to hemorrhage and necrosis in multiple organs.

At 10 mg/kg/day, formulation 4 was clearly the least well tolerated as evidenced by 100% moribundity and severe test article-related clinical signs prior to sacrifice including decreases in locomotor activity, ataxia, emesis (with or without feed, blood and/or compound), salivation, inappetence, reduced feces, pale and/or thin appearance, cold to touch, ptosis, fecal changes (diarrhea, soft, mucoid and/or reduced feces) and body weight loss (up to 15% body weight loss compared to baseline following 3 doses). Formulation 1 was also not tolerated based on moribundity in one animal, clinical signs similar to those observed in formulation 4 and body weight loss up to 7% in the dogs that survived until study termination. Formulations 2, 3 and 5 appeared to be better tolerated with all animals surviving the 1-week treatment period and with clinical signs generally less severe than those described above. Body weight losses were also minimal, ranging from 2-5% for formulation 2, 1-7% for formulation 3 and 0-5% for formulation 5.

At 25 mg/kg/day, test article-related clinical signs were noted across all dosing formulations and included decreases in locomotor activity, ataxia, ptosis, inappetence, reduced feces, emesis (with or without feed, blood and/or compound), and fecal changes. Pale or thin appearance, cold to touch and/or ataxia was noted in all formulation groups except formulation 4 since these animals were sacrificed prior to the onset of these signs. Moreover, excessive body weight loss was observed at 25 mg/kg/day in all dosing formulations by day 7 and ranged from 12-14% (formulation 1), 14% (formulation 2), 15-18% (formulation 3), and 9-12% (formulation 5) compared to baseline.

The onset of inappetence (defined as ≦50% food consumed), and emetic and fecal changes generally began on days 3 or 4 while the decreases in motor abilities and alterations in appearance (thin, cold, pale) generally began on day 5 or thereafter. The only clinical sign observed on day 1 was emesis in the animals receiving formulation 4 at 25 mg/kg/day.

Examinations

A summary of test article-related mortality, clinical signs and body weight are presented in Table 5, Table 6, Table 7, Table 8, Table 9 and Table 10.

(a)

TABLE 5
Summary of mortality data at 10 mg/kg/day

| | Formulation/group | | | | |
|---|---|---|---|---|---|
| | 1 | 2 | 3 | 4 | 5 |
| Moribundity | 1/3 | 0/3 | 0/3 | 3/3 | 0/3 |

(b)

TABLE 6
Summary of mortality data at 25 mg/kg/day

| | Formulation/group | | | | |
|---|---|---|---|---|---|
| | 1 | 2 | 3 | 4 | 5 |
| Moribundity | 3/3 | 2/3 | 3/3 | 3/3 | 1/3 |

(c)

TABLE 7
Summary of clinical signs data at 10 mg/kg/day

| | Formulation/group | | | | |
|---|---|---|---|---|---|
| | 1 | 2 | 3 | 4 | 5 |
| Pale appearance and/or cold to | 1 | 0 | 0 | 2 | 0 |
| touch | | | | | |
| Thin appearance | 3 | 2 | 0 | 3 | 0 |
| Excessive drinking | 0 | 0 | 0 | 1 | 0 |
| Decreases in locomotor activity | 1 | 1 | 1 | 3 | 2 |
| Ataxia | 1 | 0 | 0 | 2 | 0 |
| Fecal changes (soft, diarrhea | 3 | 2 | 2 | 3 | 1 |
| and/or mucoid) | | | | | |
| Feces blood | 0 | 0 | 1 | 0 | 0 |
| Feces reduced | 3 | 2 | 3 | 3 | 2 |
| 50% food consumption | 2 | 1 | 3 | 3 | 2 |
| 25% food consumption | 3 | 1 | 2 | 2 | 1 |
| 0% food consumption | 1 | 0 | 0 | 1 | 0 |
| Salivation | 1 | 0 | 0 | 3 | 1 |
| Reddened skin and/or sclera | 0 | 1 | 1 | 1 | 0 |
| Emesis (with or without feed, blood | 3 | 2 | 3 | 3 | 1 |
| and/or compound) | | | | | |
| Labored respiration | 1 | 0 | 0 | 1 | 0 |

(d)

TABLE 8
Summary of clinical signs data at 25 mg/kg/day

| | Formulation/group | | | | |
|---|---|---|---|---|---|
| | 1 | 2 | 3 | 4 | 5 |
| Pale appearance and/or cold to | 3 | 1 | 3 | 0 | 0 |
| touch | | | | | |
| Thin appearance | 3 | 3 | 3 | 0 | 2 |
| Dehydration | 0 | 0 | 1 | 0 | 0 |
| Decreases in locomotor activity | 3 | 3 | 3 | 3 | 3 |
| Ataxia | 1 | 2 | 3 | 0 | 1 |
| Ptosis | 0 | 2 | 2 | 1 | 1 |
| Muscle tremors | 0 | 0 | 0 | 0 | 1 |
| Reddened sclera and skin | 0 | 1 | 1 | 0 | 0 |
| Fecal changes (soft, diarrhea | 3 | 2 | 2 | 3 | 3 |
| and/or mucoid) | | | | | |
| Feces blood | 1 | 0 | 1 | 0 | 0 |
| Feces reduced | 3 | 3 | 3 | 2 | 3 |
| 50% food consumption | 1 | 3 | 1 | 1 | 2 |
| 25% food consumption | 3 | 3 | 3 | 2 | 3 |
| 0% food consumption | 1 | 3 | 2 | 0 | 3 |
| Salivation | 3 | 1 | 2 | 2 | 1 |
| Emesis (with or without feed, blood | 3 | 3 | 3 | 3 | 3 |
| and/or compound) | | | | | |

(e)

(f)

TABLE 9
Test article-related body weight loss in
animals sacrificed early

| Formulation/group | Observation | Body weight | Body weight |
|---|---|---|---|
| Dose (mg/kg) | period (day) | (kg) | % gain (to D1) |
| 1 (10) | 1 | 7.8 | — |
| | 4 | 7.6 | 2% loss |
| | 7 | 6.9 | 12% loss |
| 1 (25) | 1 | 9.8 | — |
| | 4 | 9.3 | 5% loss |
| | 7 | 8.6 | 12% loss |
| 1 (25) | 1 | 9.1 | — |
| | 4 | 8.5 | 7% loss |
| | 7 | 7.8 | 14% loss |
| 1 (25) | 1 | 9.2 | — |
| | 4 | 8.5 | 8% loss |
| | 6 | 7.8 | 15% loss |
| 2 (25) | 1 | 8.3 | — |
| | 4 | 7.8 | 6% loss |
| | 7 | 7.1 | 14% loss |
| 2 (25) | 1 | 8.8 | — |
| | 4 | 8.3 | 6% loss |
| | 7 | 7.6 | 14% loss |
| 3 (25) | 1 | 8.9 | — |
| | 4 | 8.3 | 7% loss |
| | 6 | 7.5 | 16% loss |
| 3 (25) | 1 | 8.7 | — |
| | 4 | 8.0 | 8% loss |
| | 7 | 7.4 | 15% loss |
| 3 (25) | 1 | 10.1 | — |
| | 4 | 9.3 | 8% loss |
| | 7 | 8.3 | 18% loss |
| 4 (10) | 1 | 9.0 | — |
| | 4 | 8.9 | 1% loss |
| | 7 | 8.0 | 11% loss |
| 4 (10) | 1 | 8.6 | — |
| | 4 | 7.6 | 15% loss |
| 4 (10) | 1 | 7.9 | — |
| | 4 | 7.3 | 8% loss |
| | 7 | 6.7 | 15% loss |
| 4 (25) | 1 | 8.0 | — |
| | 4 | 7.7 | 4% loss |
| 4 (25) | 1 | 8.9 | — |
| | 4 | 8.5 | 4% loss |
| 4 (25) | 1 | 9.7 | — |
| | 4 | 9.3 | 4% loss |
| 5 (25) | 1 | 7.7 | — |
| | 4 | 7.5 | 3% loss |
| | 7 | 6.8 | 12% loss |

(g)

(h)

TABLE 10
Test article-related body weight loss in
animals that survived until study termination

| Formulation | Observation | Body weight | Body weight |
|---|---|---|---|
| Dose (mg/kg) | period (day) | (kg) | % gain (to D1) |
| 1 (10) | 1 | 8.6 | — |
| | 4 | 8.0 | 7% loss |
| | 7 | 8.2 | 5% loss |
| 1 (10) | 1 | 8.0 | — |
| | 4 | 7.8 | 3% loss |
| | 7 | 7.6 | 5% loss |
| 2 (10) | 1 | 8.9 | — |
| | 4 | 8.7 | 2% loss |
| | 7 | 8.6 | 2% loss |
| 2 (10) | 1 | 8.2 | — |
| | 4 | 7.8 | 5% loss |
| | 7 | 8.0 | 2% loss |
| 2 (10) | 1 | 9.8 | — |
| | 4 | 9.6 | 2% loss |
| | 7 | 9.6 | 2% loss |
| 2 (25) | 1 | 8.6 | — |
| | 4 | 8.2 | 5% loss |
| | 7 | 7.4 | 14% loss |
| 3 (10) | 1 | 7.6 | — |
| | 4 | 7.3 | 4% loss |
| | 7 | 7.2 | 5% loss |
| 3 (10) | 1 | 8.1 | — |
| | 4 | 7.9 | 1% loss |
| | 7 | 7.7 | 5% loss |
| 3 (10) | 1 | 8.2 | — |
| | 4 | 7.8 | 5% loss |
| | 7 | 7.6 | 7% loss |
| 5 (10) | 1 | 8.5 | — |
| | 4 | 8.4 | 1% loss |
| | 7 | 8.5 | 0 |
| 5 (10) | 1 | 8.3 | — |
| | 4 | 8.3 | 0 |
| | 7 | 7.9 | 5% loss |
| 5 (10) | 1 | 9.3 | — |
| | 4 | 9.0 | 3% loss |
| | 7 | 9.0 | 3% loss |
| 5 (10) | 1 | 8.9 | — |
| | 4 | 8.8 | 1% loss |
| | 7 | 8.1 | 9% loss |
| 5 (10) | 1 | 8.2 | — |
| | 4 | 7.9 | 4% loss |
| | 7 | 7.2 | 12% loss |

Toxicokinetic Assessments

Mean toxicokinetic parameters are presented in Table 11 for day 1 and Table 12 for day 7. The tₘₐₓ generally occurred at 0.5 hours postdose at both dose levels for formulations 1, 2 and 4 except for formulation 4, on day 1 at 25 mg/kg/day. The tₘₐₓ for formulations 3 and 5 was generally 0.5 to 2 hours postdose on both days at both dose levels and is consistent with the slow release component of the formulation.

At 10 mg/kg/day, a slight tendency towards accumulation was detected for formulations 1, 2, 3 and 5 from day 1 to day 7.

(a)

TABLE 11
Mean toxicokinetic parameters of
Zoledronic acid on day 1

| | 10 mg/kg/day | | | | |
|---|---|---|---|---|---|
| | Formulation 1 | Formulation 2 | Formulation 3 | Formulation 4 | Formulation 5 |
| | N = 3 | N = 3 | N = 3 | N = 3 | N = 3 |
| tₘₐₓ(hrs) | 0.5 to 0.5 | 0.5 to 0.5 | 0.5 to 2 | 0.5 to 0.5 | 0.5 to 2 |
| Cₘₐₓ(ng/mL) | 902.3 | 463.7 | 284.0 | 4437.7 | 454.3 |
| Cₘₐₓ/dose | 90.2 | 46.4 | 28.4 | 444.0 | 45.4 |
| [(ng/mL)/(mg/kg/day)] | | | | | |
| AUC(0-24 h) (ng · hrs/mL) | 1254.0 | 631.0 | 592.5 | 6949.0 | 954.0 |
| AUC(0-24 h)/dose | 125.4 | 63.1 | 59.3 | 695.0 | 95.4 |
| [(ng · hrs/mL)/(mg/kg/day)] | | | | | |
| | 25 mg/kg/day | | | | |
| | Formulation 1 | Formulation 2 | Formulation 3 | Formulation 4 | Formulation 5 |
| | N = 3 | N = 3 | N = 3 | N = 3 | N = 2 |
| tₘₐₓ(hrs) | 0.5 to 0.5 | 0.5 to 0.5 | 2 to 2 | 0.5 to 2 | 2 to 2 |
| Cₘₐₓ(ng/mL) | 3102.3 | 2233.3 | 1345.7 | 7923.3 | 1146.0 |
| Cₘₐₓ/dose | 124.1 | 89.3 | 53.8 | 317.0 | 45.8 |
| [(ng/mL)/(mg/kg/day)] | | | | | |
| AUC(0-24 h) (ng · hrs/mL) | 7139.0 | 4435.0 | 4010.0 | 20065.0 | 2046.4 |
| AUC(0-24 h)/dose | 285.6 | 177.4 | 160.0 | 803.0 | 81.9 |
| [(ng · hrs/mL)/(mg/kg/day)] | | | | | |

(b)

TABLE 12
Mean toxicokinetic parameters of Zoledronic
acid on day 7

| | 10 mg/kg/day | | | | |
|---|---|---|---|---|---|
| | Formulation 1 | Formulation 2 | Formulation 3 | | Formulation 5 |
| | N = 2 | N = 3 | N = 3 | Formulation 4 | N = 3 |
| tₘₐₓ(hrs) | 0.5 to 0.5 | 0.5 to 0.5 | 2 to 2 | | 0.5 to 2 |
| Cₘₐₓ(ng/mL) | 710.0 | 691.3 | 1161.7 | | 917.7 |
| Cₘₐₓ/dose | 71.0 | 69.1 | 116.2 | | 91.8 |
| [(ng/mL)/(mg/kg/day)] | | | | | |
| AUC(0-24 h) (ng · hrs/mL) | 1279 | 1294 | 3809.0 | | 2544.0 |
| AUC(0-24 h)/dose | 127.9 | 129.4 | 381.0 | | 254.0 |
| [(ng · hrs/mL)/(mg/kg/day)] | | | | | |
| | 25 mg/kg/day | | | | |
| | | Formulation 2 | | | Formulation 5 |
| | Formulation 1 | N = 1 | Formulation 3 | Formulation 4 | N = 2 |
| tₘₐₓ(hrs) | | 0.5 to 0.5 | | | 0.5 to 2 |
| Cₘₐₓ(ng/mL) | | 5926.0 | | | 3213.0 |
| Cₘₐₓ/dose | | 237.0 | | | 128.5 |
| [(ng/mL)/(mg/kg/day)] | | | | | |
| AUC(0-24 h) (ng · hrs/mL) | | 11888.0 | | | 11407.0 |
| AUC(0-24 h)/dose | | 476.0 | | | 456.3 |
| [(ng · hrs/mL)/(mg/kg/day)] | | | | | |

Conclusion

The Example demonstrates that there can be significant gastric absorption of zoledronic acid with tolerable side effects in the gastro-intestinal tract using formulations of the present invention with lipophilic bioavailability enhancers and solubilizers such as CAPMUL PG-8 and VITAMIN E-TPGS.

1. A pharmaceutical formulation comprising an oral dosage form containing a bisphosphonic acid or a salt thereof and an inactive ingredient selected from: an ester of a medium chain fatty acid, or a lipophilic polyethylene glycol ester, said inactive ingredient having a hydrophilic-lipophilic balance (HLB) of from about 1 to about 30.

2. A pharmaceutical formulation according to claim 1 wherein said bisphosphonic acid or salts thereof is a bone resorption inhibitor.

3. A pharmaceutical formulation according to claim 1 wherein said bone resorption inhibitor is useful in treating or preventing osteoporosis or diseases related to irregular osteoclast activity.

4. A pharmaceutical formulation according to claim 1 wherein said bisphosphonic acid or a salt thereof may be selected from the group consisting of of ibandronate, alendronate, etidronate, risedronate, and tiludronate or a salt thereof.

5. A pharmaceutical formulation according to claim 1 wherein said bisphosphonic acid or a salt thereof is zoledronic acid or a salt thereof.

6. A pharmaceutical formulation according to claim 1 wherein said inactive ingredient is a propylene glycol monoester of medium chain fatty acids.

7. A pharmaceutical formulation according to claim 6 wherein said inactive ingredient has an HLB of 4.4.

8. A pharmaceutical formulation according to claim 1 wherein said inactive ingredient is D-alpha-tocopheryl polyethylene glycol 1000 succinate.

9. A pharmaceutical formulation according to claim 1 wherein said inactive ingredient is a combination of is a propylene glycol monoester of medium chain fatty acids (primarily caprylic acid) and D-alpha-tocopheryl polyethylene glycol 1000 succinate.

10. A pharmaceutical formulation according to claim 1 wherein said dose of bisphosphonic acid or salt thereof is in the range of from about 0.01 mg/kg to about 500 mg/kg.

11. A pharmaceutical formulation according to claim 1 wherein said dose of bisphosphonic acid or salt thereof is in the range of from about 0.1 mg/kg to about 200 mg/kg.

12. A pharmaceutical formulation according to claim 1 wherein said dose of bisphosphonic acid or salt thereof is in the range of from about 0.2 mg/kg to about 100 mg/kg.

13. A method of treatment comprising administering an oral dosage form according to claim 1 in order to provide increased bioavailability or increased tolerability of said bisphosphonic acid or salt thereof.

14. A method according to claim 13 wherein said increased bioavailability is measured as increased absolute bioavailability.

15. A method according to claim 14 wherein said absolute bioavailability is in the range of from about 1% to about 50%.

16. A method according to claim 14 wherein said absolute bioavailability is in the range of from about 2.5% to about 30%.

17. A method according to claim 14 wherein said absolute bioavailability is in the range of from about 7.5% to about 20%.

18. A method according to claim 14 wherein said increased bioavailability is measured in said subject as a blood level Cmax in the range of from about 1 to about 16,000 ng/mL.

19. A method according to claim 14 wherein said increased bioavailability is measured in said subject as a blood level Cmax in the range of from about 10 to about 8,000 ng/mL.

20. A method according to claim 14 wherein said increased bioavailability is measured in said subject as a blood level AUC (0-24Hr) in the range of from about 100 to about 40,000 ng/hr/mL.

21. A method according to claim 14 wherein said increased bioavailability is measured in said subject as a blood level AUC (0-24Hr) in the range of from about 100 to about 20,000 ng/hr/mL.

22. A method according to claim 14 wherein said increased tolerability is measured as reduced gastrointestinal toxicity.

23. A method of treatment comprising administering a dosage form according to claim 1 in order to provide increased bioavailability and increased tolerability of said bisphosphonic acid or salts thereof.

24. A process for preparing a formulation as defined in claim 1 comprising: suspending the bisphosphonic acid or a salt thereof in the inactive ingredient to produce a dispersion; and encapsulating the dispersion.

25. A process according to claim 23 wherein the inactive ingredient is pre-heated prior to suspending the bisphosphonic acid or salt thereof.

26. A process according to claim 23 wherein the dispersion is encapsulated in gelatin capsules.

27. A pharmaceutical formulation according to claim 1 wherein said inactive ingredient is caprylic acid.
